(19) United States
(12) Patent Application Publication
(10) Pub. No.: US 2006/0173042 A1
(43) Pub. Date: Aug. 3, 2006

(54) Process for the preparation of fexofenadine

(76) Inventors: Mukesh Kumar Sharma, Haryana (IN); Chandra Has Khanduri, Haryana (IN); Naresh Kumar, Chandigarh (IN)

Correspondence Address: RANBAXY INC., 600 COLLEGE ROAD EAST, SUITE 2100, PRINCETON, NJ, 08540, US

(21) Appl. No.: 10/538,956

(22) PCT Filed: Dec. 15, 2003
(86) PCT No.: PCT/IB03/05994

§ 371 Date: Feb. 28, 2006

(30) Foreign Application Priority Data
Dec. 16, 2002 (IN) 1262/DEL/2002

(51) Int. Cl. A61K 31/445; C07D 211/34; C07C 59/76

(52) U.S. Cl. 514/317; 546/238; 562/460

(57) ABSTRACT

The present invention relates to a process for the preparation of cyclopropyl keto a,a-dimethylphenyl acetic acid of structural Formula I, and to the use of this compound as an intermediate for the preparation of an antihistamine, fexofenadine.

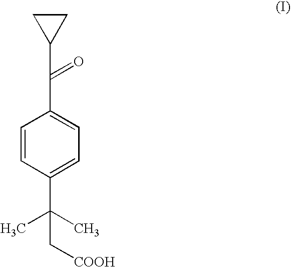

### FIELD OF THE INVENTION

The field of the invention relates to a process for the preparation of cyclopropyl keto α,α-dimethylphenyl acetic acid of structural Formula I, and to the use of this compound as an intermediate for the preparation of an antihistamine, fexofenadine.

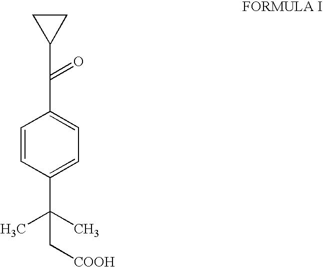

### BACKGROUND OF THE INVENTION

Chemically, fexofenadine is 4[1-hydroxy-4-[4-(hydroxydiphenylmethyl)-1-piperidinyl]butyl]-α,α-dimethylbenzene acetic acid of structural Formula II, and is known 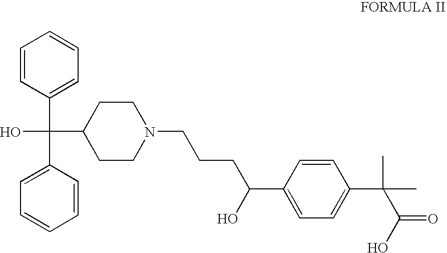
from U.S. Pat. No. 4,254,129. It is one of the most widely used antihistamines for the treatment of allergic reactions.

In general, the synthetic approach reported in the literature for the preparation of cyclopropyl keto α,α-dimethylphenyl acetic acid involves the treatment of the corresponding alcohol of Formula III with a conventional oxidizing agent (EP 705245, EP 1178041, and WO 95/00480). The oxidation can be done in either two steps or a single step.

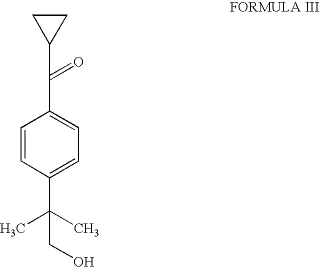

The oxidizing agents reported in the literature for such reactions are ruthenium chloride/sodium periodate in solvents like acetonitrile or carbon tetrachloride, fuming nitric acid in acetic acid, dimethylsulphoxide/oxalyl chloride/triethylamine, Dess Martin reagent, chromium 4-oxide, nickel peroxide, sodium dichromate, and manganese dioxide.

The prior art approach is not suitable from commercial point of view because it is not environmental friendly, expensive and requires cumbersome work up process. Most of the reagents are disadvantageous as these results in run away reactions, which lower the yields thus making the approach commercially difficult to implement.

Thus, the present invention provides a process for the preparation of cyclopropyl keto α,α-dimethylphenyl acetic acid which does not require the use of any organic solvent during oxidation, rather uses water. The process of the present invention reduces the impurities, eliminates the costly and time consuming purification step as it provides the fexofenadine which does not require any further purification.

### SUMMARY OF THE INVENTION

In one general aspect there is provided a process for the preparation of cyclopropyl keto α,α-dimethylphenyl acetic acid. The process includes treating 4-(cyclopropyloxomethyl)-2,2-dimethylphenethyl alcohol with a hydroxide of an alkali metal; adding oxidizing agent followed by aqueous acidic work up; and isolating the cyclopropyl keto α,α-dimethylphenyl acetic acid.

The process may include drying the product obtained.

The hydroxide of an alkali metal may be lithium hydroxide, sodium hydroxide, or potassium hydroxide. In particular, the hydroxide is sodium hydroxide.

In one general aspect organic solvent may be added to the reaction mixture after the oxidation reaction is complete and filtered to remove inorganic solids before the aqueous acidic work up.

The organic solvent may be one or more of ketone, chlorinated solvent, or mixtures thereof. The ketone may include one or more of acetone, 2-butanone, and 4-methylpentan-2-one. The chlorinated solvent may include one or more of dichloromethane, dichloroethane, and chloroform.

In another general aspect the filtrate obtained after removal of the inorganic solids may be washed with one or more solvent to remove non-acidic impurities.

The solvent may be one or more of a chlorinated solvent, or mixtures thereof. The chlorinated solvent may include one or more of dichloromethane, dichloroethane, and chloroform.

In another general aspect there is provided a process for the preparation of fexofenadine from the cyclopropyl keto α,α-dimethylphenyl acetic acid.

The details of one or more embodiments of the inventions are set forth in the description below. Other features, objects and advantages of the inventions will be apparent from the description and claims.

### DETAILED DESCRIPTION OF THE INVENTION

The inventors have developed an efficient process for the preparation of cyclopropyl keto α,α-dimethylphenyl acetic acid, by treating the 4-(cyclopropyloxomethyl)-2,2-dimethylphenethyl alcohol with a hydroxide of an alkali metal, adding oxidizing agent followed by aqueous acidic work up and isolating the cyclopropyl keto α,α-dimethylphenyl acetic acid.

In general, a solution of a hydroxide of an alkali metal may be prepared by dissolving in water and treating the 4-(cyclopropyloxomethyl)-2,2-dimethylphenethyl alcohol with said solution. Alternatively, such a solution may be prepared in any solvent in which the hydroxide of an alkali metal is soluble, including, for example, lower alkanols, ketones, water and mixtures thereof.

The hydroxide of an alkali metal includes any hydroxide, including, for example, lithium hydroxide, sodium hydroxide, and potassium hydroxide.

In general, the 4-(cyclopropyloxomethyl)-2,2-dimethylphenethyl alcohol may be treated with a hydroxide of an alkali metal at room temperature, and the oxidizing agent may be added in small lots.

The oxidizing agent includes any oxidizing agent which is capable of carrying out the oxidation of the 4-(cyclopropyloxomethyl)-2,2-dimethylphenethyl alcohol, including, for example, potassium permanganate.

In general, after the oxidation reaction is complete, the reaction mass is acidified and the precipitated product is filtered. The reaction mass may be acidified with any acid, including, for example, hydrochloric acid. The product may be isolated from the solution by a technique which includes, for example, filtration, filtration under vacuum, decantation, and centrifugation.

The product may be further or additionally dried to achieve the desired moisture values. For example, the product may be further or additionally dried in a tray drier, dried under vacuum and/or in a Fluid Bed Drier.

In another aspect, organic solvent may be added to reaction mixture after the oxidation reaction is complete and filtered to remove inorganic solids before the aqueous acidic work up.

In general, after the addition of organic solvent to the reaction mass, inorganic solids resulting from the oxidation reaction precipitate out which can be filtered easily by conventional techniques.

The term “organic solvent” includes any solvent or solvent mixture which is capable of precipitating inorganic solids, including, for example, ketones, chlorinated solvents, and mixtures thereof. Examples of ketones include solvents such as acetone, 2-butanone, and 4-methylpentan-2-one. A suitable chlorinated solvent includes one or more of dichloromethane, dichloroethane, and chloroform. Mixtures of all of these solvents are also contemplated.

In another aspect, the filtrate obtained after removal of the inorganic solids may be washed with one or more solvent to remove non-acidic impurities.

The term “solvent” includes any solvent or solvent mixture which is capable of removing the non-acidic impurities, including, for example, chlorinated solvents. A suitable chlorinated solvent includes one or more of dichloromethane, dichloroethane, and chloroform. Mixtures of all of these solvents are also contemplated.

In general, after separating the inorganic solids by filtration, the two layers can be separated. The aqueous layer containing the product can be successively washed with a chlorinated hydrocarbon in order to remove the non-acidic impurities generated during the reaction. After removal of the non-acidic impurities, the aqueous layer is acidified to get the desired product.

The cyclopropyl keto α,α-dimethylphenyl acetic acid so obtained may be converted to fexofenadine or a pharmaceutically acceptable salt thereof by the methods known in the literature (EP 705245; 1178041 and WO 95/00480). The conversion to fexofenadine includes the steps of hydrolysis, condensation with azacyclonol, and reduction. The azacyclonol may be prepared by the methods known in the literature.

The present invention is further illustrated by the following examples which are provided merely to be exemplary of the inventions and is not intended to limit the scope of the invention. Certain modifications and equivalents will be apparent to those skilled in the art and are intended to be included within the scope of the present invention.

### EXAMPLE

### Preparation of cyclopropyl keto α,α-dimethylphenyl acetic acid

To a solution of sodium hydroxide (11.5 g) in water, 4-(cyclopropyloxomethyl)-2,2-dimethylphenethyl alcohol (125 g) was added at room temperature to get a suspension. To the above suspension, solid potassium permanganate was added in small lots over a period of 4-5 hours at room temperature. After the completion of reaction, acetone (1 ml) was added, and manganese dioxide so formed was filtered. The filtrate was washed with dichloromethane (25 ml+12.5 ml) to remove non-acidic impurities. The product was isolated from the aqueous layer by acidification with hydrochloric acid to yield 23.7 g material of good purity.

While several particular forms of the inventions have been described, it will be apparent that various modifications and combinations of the inventions detailed in the text can be made without departing from the spirit and scope of the inventions. Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

We claim:

1. A process for the preparation of cyclopropyl keto α, α-dimethylphenyl acetic acid of Formula I, 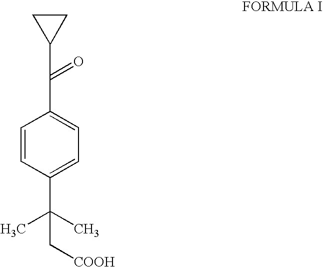
the process comprising treating 4-(cyclopropyloxomethyl)-2,2-dimethylphenethyl alcohol of Formula III, 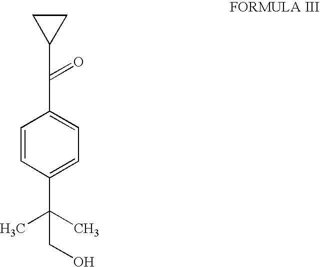
with a hydroxide of an alkali metal; adding oxidizing agent followed by aqueous acidic work up; and isolating the cyclopropyl keto α,α-dimethylphenyl acetic acid.

2. The process of claim 1, wherein the hydroxide of an alkali metal is lithium hydroxide, sodium hydroxide, and potassium hydroxide.

3. The process of claim 2, wherein the hydroxide of an alkali metal is sodium hydroxide.

4. The process of claim 1, wherein the oxidizing agent is potassium permanganate.

5. The process of claim 1, wherein the oxidizing agent is added in small lots.

6. A process for the preparation of cyclopropyl keto α,α-dimethylphenyl acetic acid of Formula I, 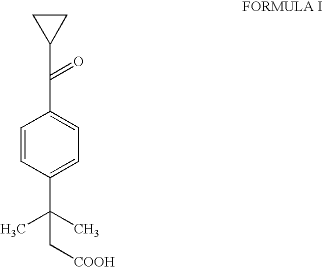
the process comprising treating 4-(cyclopropyloxomethyl)-2,2-dimethylphenethyl alcohol of Formula III, 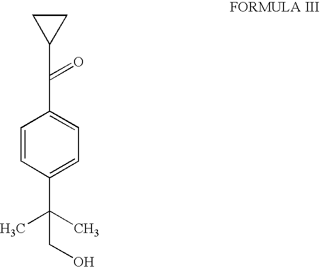
with a hydroxide of an alkali metal; adding oxidizing agent; adding organic solvent followed by aqueous acidic work up; and isolating the cyclopropyl keto α,α-dimethylphenyl acetic acid.

7. The process of claim 6, wherein the hydroxide of an alkali metal is lithium hydroxide, sodium hydroxide, and potassium hydroxide.

8. The process of claim 7, wherein the hydroxide of an alkali metal is sodium hydroxide.

9. The process of claim 6, wherein the oxidizing agent is potassium permanganate.

10. The process of claim 6, wherein the oxidizing agent is added in small lots.

11. The process of claim 6, wherein the organic solvent comprises one or more of chlorinated hydrocarbon, ketone, or mixtures thereof.

12. The process of claim 11, wherein the ketone comprises one or more of acetone, methyl ethyl ketone, and methyl isobutyl ketone.

13. The process of claim 12, wherein the ketone is acetone.

14. The process of claim 11, wherein the chlorinated hydrocarbon comprises one or more of dichloromethane, chloroform, and 1,2-dichloroethane.

15. The process of claim 6, further comprising removing precipitated inorganic solids after adding organic solvent.

16. The process of claim 15, wherein the inorganic solids are removed by filtration.

17. The process of claim 16, further comprising washing filtrate with one or more of a chlorinated solvent after removal of the inorganic solids.

18. The process of claim 17, wherein the chlorinated hydrocarbon comprises one or more of dichloromethane, chloroform, and 1,2-dichloroethane.

19. A process for the preparation of fexofenadine of Formula II or a pharmaceutically acceptable salt thereof, 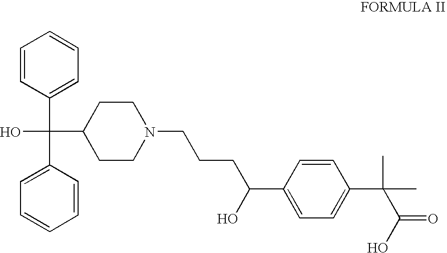
the process comprising hydrolyzing the cyclopropyl keto α, α-dimethylphenyl acetic acid of Formula I prepared by the process of claim 1 or 6, condensing with 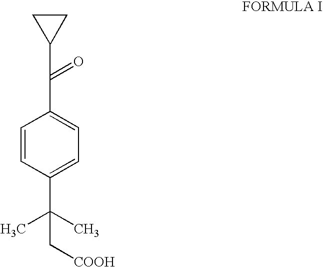
azacyclonol, and reducing.

20. A method of treating allergic reactions in a patient in need thereof, the method comprising providing a dosage form to said patient that includes fexofenadine hydrochloride prepared by the process of claim 19.
